(19) United States
(12) Patent Application Publication
(10) Pub. No.: US 2024/0082428 A1
(43) Pub. Date: Mar. 14, 2024

(54) GENETIC THERAPY FOR GLAUCOMA AND OPTIC NEUROPATHIES

(71) Applicants: The Board of Trustees of the Leland Stanford Junior University, Stanford, CA (US); The United States Government as Represented by The Department of Veterans Affairs, Washington, DC (US)

(72) Inventors: Jeffrey L. Goldberg, Menlo Park, CA (US); Kun-Che Chang, Redwood City, CA (US)

(21) Appl. No.: 18/271,631

(22) PCT Filed: Feb. 2, 2022
(86) PCT No.: PCT/US2022/014949

§ 371 Date: Jul. 10, 2023

Related U.S. Application Data
(60) Provisional application No. 63/145,161, filed on Feb. 3, 2021.

(51) Int. Cl. A61K 48/00; C07K 14/47; C12N 15/113

(52) U.S. Cl. CPC A61K 48/005; C07K 14/4702; C12N 15/113; C12N 2310/11; C12N 2710/16143; C12N 2730/10143; C12N 2750/14143

(57) ABSTRACT

Methods and compositions are provided for the treatment of glaucoma and other optic neuropathies.

### CROSS REFERENCE TO RELATED APPLICATION

The present application is a 371 and claims the benefit of PCT Application No. PCT/US2022/014949, filed Feb. 2, 2022, which claims the benefit of and priority to U.S. Provisional Patent Application No. 63/145,161, filed Feb. 3, 2021, which applications are incorporated herein by reference in their entirety.

### INCORPORATION BY REFERENCE OF SEQUENCE LISTING

A Sequence Listing is provided herewith as a Sequence Listing text, STAN-1814_SEQ_LISTING_ST25 created on Jul. 10, 2023, created and having a size of 3,541 bytes. The contents of the Sequence Listing text are incorporated herein by reference in their entirety.

### BACKGROUND

The forms of glaucoma are a group of optic neuropathies characterized by progressive degeneration of retinal ganglion cells. These are central nervous system neurons that have their cell bodies in the inner retina and axons in the optic nerve. Degeneration of these nerves results in cupping, a characteristic appearance of the optic disc and visual loss. The biological basis of glaucoma is poorly understood and the factors contributing to its progression have not been fully characterized.

Glaucoma affects more than 70 million people worldwide with approximately 10% being bilaterally blind, making it the leading cause of irreversible blindness in the world. Glaucoma can remain asymptomatic until it is severe, resulting in a high likelihood that the number of affected individuals is much higher than the number known to have it. Population-level surveys suggest that only 10% to 50% of people with glaucoma are aware they have it. Forms of glaucoma can be classified into 2 broad categories: open-angle glaucoma and angle-closure glaucoma. In the United States, more than 80% of cases are open-angle glaucoma; however, angle-closure glaucoma is responsible for a disproportionate number of patients with severe vision loss. Both open-angle and angle-closure glaucoma can be primary diseases. Secondary glaucoma can result from trauma, certain medications such as corticosteroids, inflammation, tumor, or conditions such as pigment dispersion or pseudo-exfoliation.

Therapeutic methods to alleviate glaucoma are of great clinical interest and addressed by this.

### SUMMARY

Methods and compositions are provided for the treatment of glaucoma and other optic neuropathies. In such methods, an effective dose of a composition comprising a polynucleotide sequence encoding one or both of Pou2f2, or Tppp3, is administered to the individual for prevention or treatment of glaucoma or other optic neuropathies. In some embodiments the polypeptide encodes Tppp3. In some embodiments the polypeptide encodes Pou2f2. In some embodiments the polypeptide is operably linked to a promoter active in retinal cells, e.g. retinal ganglion cells. In some embodiments the Pou2f2 or Tppp3 sequence is a human Pou2f2 or human Tppp3 sequence. In some embodiments, delivery is intravitreal. In other embodiments the polynucleotide is a fragment is a fragment of one or both of Pou2f2 and Tppp3 sequences, or is complementary to one or both of Pou2f2 and Tppp3 sequences. Such polynucleotides may encode, for example, siRNA, shRNA, anti-sense RNA, guide RNAs, etc. that are specific for Pou2f2 or Tppp3.

In some embodiments, the methods provide genetic sequences encoding functional human Pou2f2 or human Tppp3 proteins for improving the growth of axons of retinal ganglion cells (RGCs) that have been damaged, or are susceptible to damage, as a result of glaucoma or other optic neuropathies, in an individual in need thereof. In some embodiments, the RGCs are human RGCs. In some embodiments the composition is formulated for injection. In some embodiments, the composition is formulated for intraocular injection, subretinal injection, intravitreal injection, periocular injection, subconjunctival injection, retrobulbar injection, intracameral injection, or sub-Tenon's injection. Alternatively the formulation is administered parenterally.

In some embodiments, a method is provided for treating or preventing glaucoma or other optic neuropathies in an individual in need thereof, the method comprising administering to the individual a therapeutically effective amount of an injectable composition, comprising, consisting or consisting essentially of: (a) one or both of a sequence encoding functional human Pou2f2 or Tppp3; or one or both of a functional human Pou2f2 or Tppp3 polypeptide; and (b) a pharmaceutically acceptable diluent, excipient, vehicle, or carrier. In some embodiments the injectable composition consists essentially of (a) one or both of a sequence encoding Pou2f2 or Tppp3; or one or both of a Pou2f2 or Tppp3 polypeptide; (b) an additional therapeutic agent; and (c) a pharmaceutically acceptable diluent, excipient, vehicle, or carrier. In some embodiments, the additional therapeutic agent is an additional agent for treating glaucoma and other optic neuropathies. In some embodiments, the additional therapeutic agent is selected from: aunorubicin, retinoic acid, 5-fluorouracil, intravitreal triamcinolone acetonide, ranibizumab, bevacizumab, dasatinib, pegaptanib sodium, N-acetyl-cysteine (NAC), pioglitazone, glucosamine, genistin, geldanamycin, fausdil, resveratrol, pentoxyfilline, dipyridamole, a corticosteroid, and an antioxidant. In some embodiments, the therapeutically effective amount is effective for promoting the growth of RGC axons.

Provided herein are data demonstrating a Pou2f2 and a Tppp3 effect on RGC differentiation and axon growth. Pou2f2 and Tppp3 overexpression or silencing alters the expression of genes associated with RGC differentiation and function. Tppp3 overexpression is shown to increase RGC axon growth, relative to controls.

### BRIEF DESCRIPTION OF DRAWINGS

FIG. 1. Identification of novel RGC genes by single cell RNA sequencing analysis. (A) Known RGC genes (Pou4f1 and Elavl4) were mainly expressed on clusters 3, 5 and 6. Pou2f2 and Tppp3 were observed in the same clusters as known RGC genes were. (B) Pou2f2 and Tppp3 expression was reduced by GDF11 treatment, which was known to suppress RGC differentiation (Pou4f1). These data suggest that these two genes are potential novel RGC markers. Red arrow indicates GDF11 treatment group at cluster 3.

FIG. 2. Pou2f2 and Tppp3 express in mouse RGC in vitro and in vivo. (A) Several RGC marker genes including Pou2f2 and Tppp3 were probed in mouse RGC by Western blot. In vivo, Pou2f2 (B) and Tppp3 (C) were detected on the ganglion cell layer and arrows indicate the Brn3a co-labeling cells. (D) Immunostaining showed that Pou2f2 expresses in the soma and neurite of RGC but Tppp3 expression is very low in the neurite of RGC, confirmed with pIll-Tubulin (E7, neuronal marker) co-labeling. (E) Pou2f2 and Tppp3 proteins can be detected in optic never and were significant reduced by optic nerve crush. (F) Tppp3 mainly expresses in the soma of RGC. DAPI indicates nucleus. These data confirm that these two genes express in RGC. Scale bars=200 μm, B, C; 50 μm, D, F.

FIG. 3. Pou2f2 and Tppp3 regulate RGC differentiation and axon outgrowth. (A) Messenger RNA of Pou2f2 starts to express on mouse retina on embryonic day 12 (E12) and keeps present at least till postnatal day 0 (P0). Total RNA of Pou2f2 expresses from E12 and is gradually decrease till P5. (B) Messenger RNA of Tppp3 starts to express on mouse retina on embryonic day 12 (E12) and keeps present at least till postnatal day 0 (P0). Total RNA of Tppp3 expresses from E12 and reach its peak at E14 and then gradually decrease till P5. (C) Exogenous expression of Pou2f2 and Tppp3 increases RGC marker Brn3a and Math5expression. (D) Genetic knockdown of Pou2f2 and Tppp3 reduces Brn3a expression. (E) Exogenous expression of Pou2f2 and Tppp3 slightly decreases and increases RGC axon outgrowth, respectively. (F) Genetic knockdown of Pou2f2 and Tppp3 slightly increases and decreases RGC axon outgrowth, respectively. These data indicate that these two genes regulate RGC differentiation. (N=3, * P<0.05, ** P<0.01, by one-way ANOVA with post-hoc t-test with Tukey correction. Means±SEMs shown.)

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any way.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in bacterial titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, siRNA, shRNA, guide RNA, anti-sense RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

By the terms “effective amount” and “therapeutically effective amount” of a formulation or formulation component is meant a sufficient amount of the formulation or component, alone or in a combination, to provide the desired effect. For example, by “an effective amount” is meant an amount of Pou2f2 or Tppp3, alone or in a combination, required to treat or prevent glaucoma or other optic neuropathies in a mammal. Ultimately, the attending physician or veterinarian decides the appropriate amount and dosage regimen.

The effective dose of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials. The agent is administered at a dosage, alone or in combination with other agents, that enhances neuron recovery while minimizing any side-effects. The effectiveness of recovery may be assessed, for example, by monitoring function of the neuron, e.g. maintenance or recovery of vision in glaucoma or other optic neuropathy patients, such as at least about 5% recovery, at least about 10% recovery, at least about 20%, at least about 30%, at least about 40%, at least about 50%, at least about 75%, at least about 85%, at least about 95% or more, e.g. assessing by conventional measures of vision or retinal structure. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% decrease in loss of function, e.g. visual acuity, relative to function measured in absence of the Pou2f2 or Tppp3 nucleic acid or polypeptide. Treatment may result in at least about a 10%, 15%, 20%, 25%, 30%, 35%, 40%, 45%, 50%, 55%, 60%, 65%, 70%, 75%, 80%, 85%, 90%, 95%, or even 100% decrease in symptoms of glaucoma or other optic neuropathies, compared to a subject that is not treated with a Pou2f2 or Tppp3 nucleic acid or polypeptide.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical formulation that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical formulation” is meant to encompass a formulation suitable for administration to a subject, such as a mammal, especially a human. In general a “pharmaceutical formulation” is sterile, and preferably free of contaminants that are capable of eliciting an undesirable response within the subject (e.g., the compound(s) in the pharmaceutical formulation is pharmaceutical grade). Pharmaceutical formulations can be designed for administration to subjects or patients in need thereof via a number of different routes of administration including oral, buccal, rectal, parenteral, intraperitoneal, intradermal, intracheal, intramuscular, subcutaneous, and the like.

As used herein, the term “administration” refers to the administration of a formulation or composition (i.e. a composition comprising a polynucleotide encoding Pou2f2 or Tppp3) to a subject or system. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Glaucoma: An eye disorder characterized by retinal ganglion cell death, excavation of the optic nerve head and gradual loss of the visual field. An abnormally high intraocular pressure is commonly known to be detrimental to the eye and is one of the main risk factors in glaucoma. In glaucoma patients, high intraocular pressure can result in degenerative changes in the retina. “Ocular hypertension” refers to clinical situation in individuals with an abnormally high intraocular pressure without any manifestation of defects in the visual field or optic nerve head. Individuals with ocular hypertension carry the risk of conversion to glaucoma with the risk being correlated to higher intraocular pressure measurements.

Glaucoma can be divided into open-angle form and the closed-angle forms and further classified into acute and chronic forms. There also is a normal-tension glaucoma. The glaucoma can be a primary or a secondary glaucoma. More than 80% of all glaucoma cases are chronic open angle glaucoma (COAG), also called primary open angle glaucoma. Any of these forms of glaucoma can be treated using the methods disclosed herein.

“Primary angle closure glaucoma” is caused by contact between the iris, trabecular meshwork, and peripheral cornea which in turn obstructs outflow of the aqueous humor from the eye. This contact between iris and trabecular meshwork (TM) may gradually damage the function of the meshwork until it fails to keep pace with aqueous production, and the pressure rises. In over half of all cases, prolonged contact between iris and TM causes the formation of synechiae (effectively “scars”). These cause permanent obstruction of aqueous outflow. In some cases, pressure may rapidly build up in the eye, causing pain and redness (symptomatic, or so-called “acute” angle closure). In this situation, the vision may become blurred, and halos may be seen around bright lights. Accompanying symptoms may include a headache and vomiting. Diagnosis can made from physical signs and symptoms: pupils mid-dilated and unresponsive to light, cornea edematous (cloudy), reduced vision, redness, and pain. However, the majority of cases are asymptomatic. Prior to the very severe loss of vision, these cases can only be identified by examination, generally by an eye care professional.

“Primary open-angle glaucoma” occurs when optic nerve damage results in a progressive loss of the visual field. Not all people with primary open-angle glaucoma have eye pressure that is elevated beyond normal. The increased pressure is caused by the blockage of the aqueous humor outflow pathway. Because the microscopic passageways are blocked, the pressure builds up in the eye and causes imperceptible very gradual vision loss. Peripheral vision is affected first, but eventually the entire vision will be lost if not treated. Diagnosis can be made by looking for cupping of the optic nerve and measuring visual field. Prostaglandin agonists work by opening uveoscleral passageways.

Other forms of glaucoma are developmental glaucoma and secondary glaucoma, which can occur after uveitis, iridocyclitis, intraocular hemorrhage, trauma, or an intraocular tumor. Any form of glaucoma can be treated using the methods disclosed herein.

The death of retinal ganglion cells occurs in glaucoma. Methods are disclosed herein for increasing the survival of retinal ganglion cells.

Members of the tubulin polymerization-promoting protein (TPPP) family are potent regulators of cell proliferation in most developmental and physiological processes. TPPP3, also known as P20, binds to tubulin, stabilizes and polymerizes microtubules, and displays microtubule-associated protein- (MAP-) like features. The sequence of human Tppp3 can be accessed at Genbank, refseq NP_057048.2 (protein) and NM_015964.4 (mRNA), for example as

SEQ ID NO: 1:
agtcccaccc gggagccggc agggagcgga gctgcggage cgcctggtct cccgcgtcca
tcggtccatt cctgcgtcgt tctgtccttc cgaacgcaca cttcaggage agccgcgagg
gtggcatggc agcgagcaca gacatggctg ggctggagga gagcttccgc aagtttgcca
tccatggtga ccccaaggcc agtgggcaag agatgaatgg caagaactgg gccaagctgt
gcaaggactg caaggtggct gacggaaagt ccgtgacagg gaccgatgtg gacatcgtct
tctccaaagt caaggggaag tctgctcggg tcatcaacta tgaggagttc aagaaggccc
tggaagagct ggcgaccaag agattcaagg ggaagagcaa ggaggaggcc ttcgatgcca
tctgccagct ggtggcaggc aaagagccag ccaatgtggg cgtcactaaa gcaaaaacag
ggggtgctgt agaccggctg acggacacca gcagatacac gggctcccac aaggagcgct
tcgatgagag cggcaagggc aagggcattg cgggacggca ggacatcctg gacgacagtg
gctacgtgag cgcctacaag aatgcaggca cctacgatgc caaggtgaag aagtgaggct
tgggaagacc gccctgccaa gtgcggctgc ccctgccaga ggctcaggcc tgggtctaag
gggcacgtgg agcaagagat cctggtcccc tccctgctgg acctgccacc cagagcttcc
tgcctagtcc cactgggctg gcccaccagg cctctgaccc aggctgctct gcggcccctt
cctcctcctc ttcctgctcc aacttctgtc cacctgggga cagtctgtgc ctgtagcctc
atgaccccaa cccagcccca ggcatggcta acccctgact gottgcctca tatttaagct
gctgctctgg ccaagtgcct aattttaacc cagacctcaa taaagacacc ttttgtacca
a
and
SEQ ID NO: 2
MAASTDMAGL EESFRKFAIH GDPKASGQEM NGKNWAKLCK DCKVADGKSV TGTDVDIVFS
KVKGKSARVI NYEEFKKALE ELATKRFKGK SKEEAFDAIC QLVAGKEPAN VGVTKAKTGG
AVDRLIDTSR YTGSHKERFD ESGKGKGIAG RQDILDDSGY VSAYKNAGTY DAKVKK

The sequence of a Tppp3 protein may be altered in various ways known in the art to generate targeted changes in sequence. The polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Pou2f2 is a homeobox-containing transcription factor of the POU domain family. The encoded protein binds the octamer sequence 5′-ATTTGCAT-3′, a common transcription factor binding site in immunoglobulin gene promoters. Several transcript variants encoding different isoforms have been found for this gene. The sequence of human Pou2f2 can be accessed at Genbank, for example Accession numbers NM_001207026.2; NM_001207025.3; NM_001247994.2; NM_002698.5 GI: 1677498157 for the mRNA; and for the protein XP_024307315.1; XP_024307314.1; XP_024307313.1; XP_011525345.2; XP_016882385.1; XP_016882384.1; XP_016882383.1; XP_016882382.1; XP_016882381.1; XP_016882380.1; XP_016882379.1; XP_016882378.1; XP_016882377.1; XP_016882376.1; XP_016882375.1 GI: 1034608229.

### Promoters and Vectors

Aspects of the present invention disclose expression cassettes and/or vectors comprising polynucleotides. Suitably, the polynucleotides can comprise promoters operably linked to the region of the polynucleotide that encodes e.g., a Pou2f2 or Tppp3 gene. Virtually any promoter capable of driving these polynucleotides can be used.

Targeted expression can be accomplished using a cell specific promoter. Examples of cell specific promoters are promoters for somatostatin, parvalbumin, GABAa6, L7, and calbindin. Other cell specific promoters can be promoters for kinases such as PKC, PKA, and CaMKII; promoters for other ligand receptors such as NMDAR1, NNIDAR2B, GIuR2; promoters for ion channels including calcium channels, potassium channels, chloride channels, and sodium channels; and promoters for other markers that label classical mature and dividing cell types, such as calretinin, nestin, and beta3-tubulin.

Specifically, where expression of a subject polynucleotide in a retinal ganglion cell is desired, a promoter of interest may be used. Promoters of interest include but are not limited to, mouse NEFH promoter, human promoter, mouse gamma-synuclein promoter, human synapsin-1 promoter, a human THY1 promoter etc.

Variants of the above discussed promoters may also be used. In some instances, a suitable variant comprises a nucleotide sequence having 60% or more, 70% or more, 75% or more, 80% or more, 85% or more, 90% or more, 95% or more, 99% or more or 100% nucleotide sequence identity to their corresponding “reference”, or wild-type, promoter. A person of skill in the art will recognize that various promoters drive expression in various cell types, and will be able to decide on which promoter to use for their desired outcome.

In some embodiments, the vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV could infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87: 2211-2215; Samulski et al, EMBO J., 1991. 10: 3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Viral., 1999. 73: 939-947).

AAV vectors may be prepared using any convenient methods. Adena-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RMLinden, C RParrish, Eds.) p 5-14, Rudder Arnold, London, U K (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAVI, AAV2, AAV3, AAV4, AAV5, AAV6, AAV7, AAV8, AAV9, AAVIO, AAVII, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

In some embodiments, a Pou2f2 or Tppp3 polynucleotide can be an antisense oligonucleotide (ODN), particularly synthetic ODN having chemical modifications from native nucleic acids, or nucleic acid constructs that express such antisense molecules as RNA. The antisense sequence is complementary to the targeted RNA, and inhibits its expression. One or a combination of antisense molecules may be administered, where a combination may comprise multiple different sequences.

Antisense molecules may be produced by expression of all or a part of the target RNA sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature that alter the chemistry of the backbone, sugars or heterocyclic bases.

Anti-sense molecules of interest include antagomir RNAs, e.g. as described by Krutzfeldt et al., supra., herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. In representative embodiments, the RNAi agent targets the precursor molecule of the Pou2f2 or Tppp3 RNA. By RNAi agent is meant an agent that modulates expression by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

In some embodiments, a Pou2f2 or Tppp3 polynucleotide binds to a class 2 CRISPR/Cas effector protein (e.g., a Cas9 protein; a type V or type VI CRISPR/Cas protein; a Cpf1 protein; etc.) and targets the complex to a specific location within a target nucleic acid, which is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

A guide RNA can be referred to by the protein to which it corresponds. For example, when the class 2 CRISPR/Cas effector protein is a Cas9 protein, the corresponding guide RNA can be referred to as a “Cas9 guide RNA.” Likewise, as another example, when the class 2 CRISPR/Cas effector protein is a Cpf1 protein, the corresponding guide RNA can be referred to as a “Cpf1 guide RNA.”

In some embodiments, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, a nucleic acid payload includes or encodes a gene editing tool (i.e., a component of a gene editing system, e.g., a site specific gene editing system such as a programmable gene editing system). For example, a nucleic acid payload can include one or more of: (i) a CRISPR/Cas guide RNA, (ii) a DNA encoding a CRISPR/Cas guide RNA, (iii) a DNA and/or RNA encoding a programmable gene editing protein such as a zinc finger protein (ZFP) (e.g., a zinc finger nuclease—ZFN), a transcription activator-like effector (TALE) protein (e.g., fused to a nuclease—TALEN), a DNA-guided polypeptide such as Natronobacterium gregoryi Argonaute (NgAgo), and/or a CRISPR/Cas RNA-guided polypeptide (e.g., Cas9, CasX, CasY, Cpf1, and the like); (iv) a DNA donor template; (v) a nucleic acid molecule (DNA, RNA) encoding a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb1 integrase, R4 integrase, lambda integrase, HK022 integrase, HP1 integrase, and the like); (vi) a DNA encoding a resolvase and/or invertase (e.g., Gin, Hin, y63, Tn3, Sin, Beta, and the like); and (vii) a transposon and/or a DNA derived from a transposon (e.g., bacterial transposons such as Tn3, Tn5, Tn7, Tn9, Tn10, Tn903, Tn1681, and the like; eukaryotic transposons such as Tc1/mariner super family transposons, PiggyBac superfamily transposons, hAT superfamily transposons, PiggyBac, Sleeping Beauty, Frog Prince, Minos, Himar1, and the like). In some cases a subject delivery molecule is used to deliver a protein payload, e.g., a gene editing protein such as a ZFP (e.g., ZFN), a TALE (e.g., TALEN), a CRISPR/Cas RNA-guided polypeptide (e.g., Cas9, CasX, CasY, Cpf1, and the like), a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb1 integrase, R4 integrase, lambda integrase, HK022 integrase, HP1 integrase, and the like), a resolvase/invertase (e.g., Gin, Hin, y63, Tn3, Sin, Beta, and the like); and/or a transposase (e.g., a transposase related to transposons such as bacterial transposons such as Tn3, Tn5, Tn7, Tn9, Tn10, Tn903, Tn1681, and the like; or eukaryotic transposons such as Tc1/mariner super family transposons, PiggyBac superfamily transposons, hAT superfamily transposons, PiggyBac, Sleeping Beauty, Frog Prince, Minos, Himar1, and the like). In some cases, the delivery molecule is used to deliver a nucleic acid payload and a protein payload, and in some such cases the payload includes a ribonucleoprotein complex (RNP).

Depending on the nature of the system and the desired outcome, a gene editing system (e.g. a site specific gene editing system such as a a programmable gene editing system) can include a single component (e.g., a ZFP, a ZFN, a TALE, a TALEN, a site-specific recombinase, a resolvase/integrase, a transpose, a transposon, and the like) or can include multiple components. In some cases a gene editing system includes at least two components. For example, in some cases a gene editing system (e.g. a programmable gene editing system) includes (i) a donor template nucleic acid; and (ii) a gene editing protein (e.g., a programmable gene editing protein such as a ZFP, a ZFN, a TALE, a TALEN, a CRISPR/Cas RNA-guided polypeptide such as Cas9, CasX, CasY, or Cpf1, and the like), or a nucleic acid molecule encoding the gene editing protein (e.g., DNA or RNA such as a plasmid or mRNA). As another example, in some cases a gene editing system (e.g. a programmable gene editing system) includes (i) a CRISPR/Cas guide RNA, or a DNA encoding the CRISPR/Cas guide RNA; and (ii) a CRISPR/CAS RNA-guided polypeptide (e.g., Cas9, CasX, CasY, Cpf1, and the like), or a nucleic acid molecule encoding the RNA-guided polypeptide (e.g., DNA or RNA such as a plasmid or mRNA). As another example, in some cases a gene editing system (e.g. a programmable gene editing system) includes (i) an NgAgo-like guide DNA; and (ii) a DNA-guided polypeptide (e.g., NgAgo), or a nucleic acid molecule encoding the DNA-guided polypeptide (e.g., DNA or RNA such as a plasmid or mRNA). In some cases a gene editing system (e.g. a programmable gene editing system) includes at least three components: (i) a donor DNA template; (ii) a CRISPR/Cas guide RNA, or a DNA encoding the CRISPR/Cas guide RNA; and (iii) a CRISPR/Cas RNA-guided polypeptide (e.g., Cas9, CasX, CasY, or Cpf1), or a nucleic acid molecule encoding the RNA-guided polypeptide (e.g., DNA or RNA such as a plasmid or mRNA).

As would be understood by one of ordinary skill in the art, a gene editing system need not be a system that ‘edits’ a nucleic acid. For example, it is well recognized that a gene editing system can be used to modify target nucleic acids (e.g., DNA and/or RNA) in a variety of ways without creating a double strand break (DSB) in the target DNA. For example, in some cases a double stranded target DNA is nicked (one strand is cleaved), and in some cases (e.g., in some cases where the gene editing protein is devoid of nuclease activity, e.g., a CRISPR/Cas RNA-guided polypeptide may harbor mutations in the catalytic nuclease domains), the target nucleic acid is not cleaved at all. For example, in some cases a CRISPR/Cas protein (e.g., Cas9, CasX, CasY, Cpf1) with or without nuclease activity, is fused to a heterologous protein domain. The heterologous protein domain can provide an activity to the fusion protein such as (i) a DNA-modifying activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity), (ii) a transcription modulation activity (e.g., fusion to a transcriptional repressor or activator), or (iii) an activity that modifies a protein (e.g., a histone) that is associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). As such, a gene editing system can be used in applications that modify a target nucleic acid in way that do not cleave the target nucleic acid, and can also be used in applications that modulate transcription from a target DNA.

For additional information related to programmable gene editing tools (e.g., CRISPR/Cas RNA-guided proteins such as Cas9, CasX, CasY, and Cpf1, Zinc finger proteins such as Zinc finger nucleases, TALE proteins such as TALENs, CRISPR/Cas guide RNAs, PAMs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; Burstein et al., Nature. 2016 Dec. 22—Epub ahead of print; Gao et al., Nat Biotechnol. 2016 Jul. 34(7):768-73; Shmakov et al., Nat Rev Microbiol. 2017 March; 15(3):169-182; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

### Polypeptides

In some embodiments, Pou2f2 or Tppp3 polypeptides may be used in place of polynucleotide sequences encoding Pou2f2 or Tppp3.

The polypeptides may be joined to a wide variety of other oligopeptides or proteins for a variety of purposes. By providing for expression of the subject peptides, various post-expression modifications may be achieved. For example, by employing the appropriate coding sequences, one may provide farnesylation or prenylation. The peptides may be PEGylated, where the polyethyleneoxy group provides for enhanced lifetime in the blood stream. The peptides may also be combined with other proteins in a fusion protein, typically where the two proteins are not normally joined, such as the Fc of an IgG isotype, which may be complement binding, with a toxin, such as ricin, abrin, diphtheria toxin, or the like, or with specific binding agents that allow targeting to specific moieties on a target cell.

The Pou2f2f or Tppp3 may be fused to another polypeptide to provide for added functionality, e.g. to increase the in vivo stability, or add to a transporter domain. For example, a stable plasma protein can extend the in vivo plasma half-life of the Pou2f2 or Tppp3 when present as a fusion, in particular wherein such a stable plasma protein is an immunoglobulin constant domain.

The Pou2f2 or Tppp3 for use in the subject methods may be produced from eukaryotic or prokaryotic cells or may be synthesized in vitro. Where the protein is produced by prokaryotic cells, it may be further processed by unfolding, e.g. heat denaturation, DTT reduction, etc. and may be further refolded, using methods known in the art. By using synthesizers, naturally occurring amino acids may be substituted with unnatural amino acids. The particular sequence and the manner of preparation will be determined by convenience, economics, purity required, and the like.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the formulations which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### Pharmaceutical Compositions

For administration to a subject such as a human or other mammal (e.g., companion, zoological or livestock animal), Pou2f2, Tppp3 or a variant thereof is desirably formulated into a pharmaceutical composition containing the active agent in admixture with one or more pharmaceutically acceptable diluents, excipients or carriers. Examples of such suitable excipients for can be found in U.S. Publication 2009/0298785 (incorporated by reference herein in its entirety), the Handbook of Pharmaceutical Excipients, 2nd Edition (1994), Wade and Weller, eds. Acceptable carriers or diluents for therapeutic use are well-known in the pharmaceutical art, and are described, for example, in Remington: The Science and Practice of Pharmacy, 20th Edition (2000) Alfonso R. Gennaro, ed., Lippincott Williams & Wilkins: Philadelphia, Pa.

The choice of pharmaceutical carrier, excipient or diluent can be selected with regard to the intended route of administration and standard pharmaceutical practice. The pharmaceutical composition can contain as, or in addition to, the carrier, excipient or diluent any suitable binder(s), lubricant(s), suspending agent(s), coating agent(s), solubilizing agent(s).

A person of ordinary skill in the art can easily determine an appropriate dosage to administer to a subject without undue experimentation. Typically, a physician will determine the actual dosage that will be most suitable for an individual subject based upon a variety of factors including the activity of the specific compound employed, the metabolic stability and length of action of the compound, the age, body weight, general health, diet, mode and time of administration, rate of excretion, drug combination, the severity of the particular condition, and the individual undergoing therapy. To determine a suitable dose, the physician or veterinarian could start doses levels lower than that required in order to achieve the desired therapeutic effect and gradually increase the dosage until the desired effect is achieved. This is considered to be within the skill of the artisan and one can review the existing literature on a specific agent to determine optimal dosing.

In some embodiments, the composition is administered in the form of a liquid (e.g., drop or spray) or gel suspension. Alternatively, the composition is applied to the eye via liposomes or infused into the tear film via a pump-catheter system. Further embodiments embrace a continuous or selective-release device, for example, membranes such as, but not limited to, those employed in the OCUSERT System (Alza Corp., Palo Alto. Calif.) in an alternative embodiment, the Pou2f2 or Tppp3 composition is contained within, carried by, or attached to a contact lens, which is placed on the eye. Still other embodiments embrace the use of the composition within a swab or sponge, which is applied to the ocular surface.

In some cases, the composition further comprises a pharmaceutically acceptable carrier, e.g., a pharmaceutically acceptable salt. Suitable ocular formulation excipients include FDA approved ophthalmic excipients, e.g., emulsions, solutions, solution drops, suspensions, and suspension drops. Other suitable classifications include gels, ointments, and inserts/implants.

Exemplary excipients for use in optimizing ocular formulations include alcohol, castor oil, glycerin, polyoxyl 35 castor oil, Tyloxapol, polyethylene glycol 8000 (PEG-8000), ethanol, glycerin, cremaphor, propylene glycol (pG), polypropylene glycol (ppG), and polysorbate 80. In some cases, citrate buffer and sodium hydroxide are included to adjust pH. Preferably, the formulation for ocular delivery of nutlin-3a comprises 5% cremaphor, 10% pG, 15% pPG, and 70% phosphate buffered saline (PBS).

In certain embodiments, the presently disclosed subject matter also includes combination therapies. Depending on the particular disease, disorder, or condition to be treated or prevented, additional therapeutic agents, which are normally administered to treat or prevent that condition, may be administered in combination with the compounds of this disclosure. These additional agents may be administered separately, as part of a multiple dosage regimen. Alternatively, these agents may be part of a single dosage form, mixed together with the Pou2f2 or Tppp3 polypeptides or polynucleotides.

By “in combination with” is meant the administration of a Pou2f2 or Tppp3 agent, or other compounds disclosed herein, with one or more therapeutic agents either simultaneously, sequentially, or a combination thereof. Therefore, a cell or a subject administered a combination of a Pou2f2 or Tppp3 agent, can receive one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the Pou2f2 or Tppp3 agent and one or more therapeutic agents are administered simultaneously, they can be administered to the cell or administered to the subject as separate pharmaceutical compositions or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered compound is not diminished by the sequential, simultaneous or separate administration of the subsequent compound(s).

Also provided are a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In some embodiments, the kits comprise one or more containers, including, but not limited to a vial, tube, ampule, bottle and the like, for containing the compound. The one or more containers also can be carried within a suitable carrier, such as a box, carton, tube or the like. Such containers can be made of plastic, glass, laminated paper, metal foil, or other materials suitable for holding medicaments.

In some embodiments, the container can hold a composition that is by itself or when combined with another composition effective for treating or preventing the condition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The presently disclosed kits or pharmaceutical systems also can include associated instructions for using the compounds for treating or preventing a neurodegenerative disease, disorder, or condition, e.g. optic neuritis, including glaucoma. In some embodiments, the instructions include one or more of the following: a description of the active compound; a dosage schedule and administration; precautions; warnings; indications; counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

### EXPERIMENTAL

### Methods

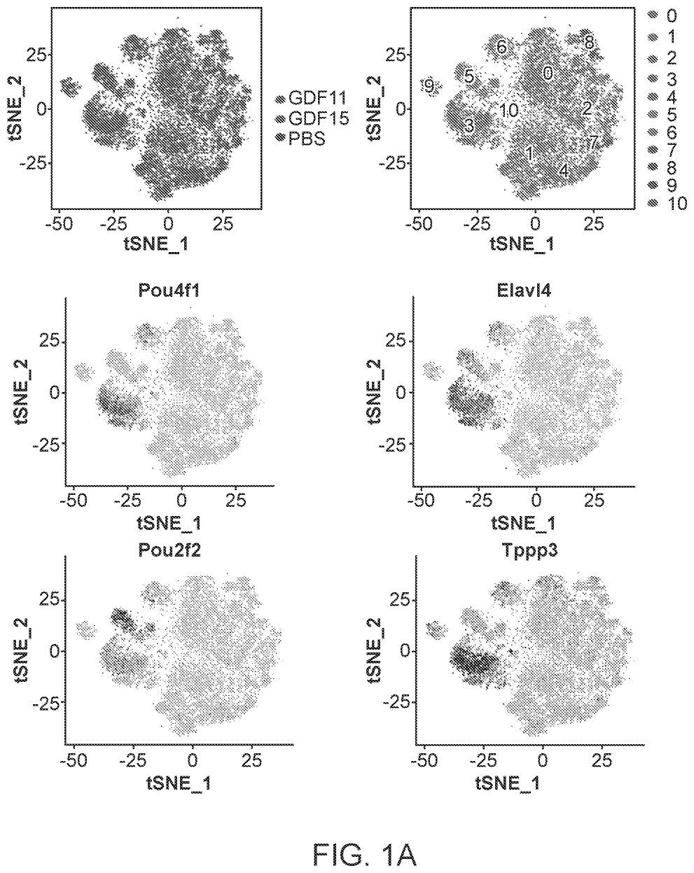

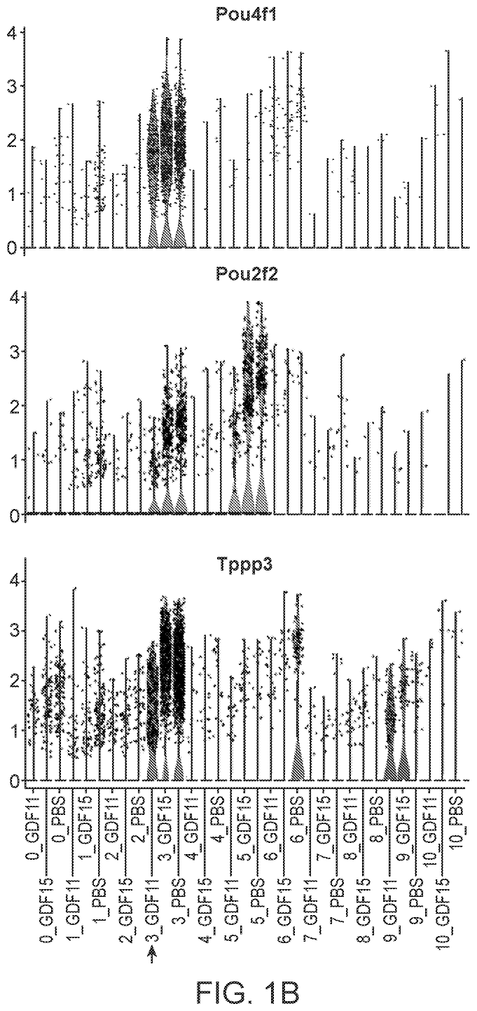

Identification of novel RGC genes by single cell RNA sequencing analysis. (A-B) E14 mouse retinas were treated with PBS or GDF11 (50 ng/ml) or GDF15 (50 ng/ml), refresh daily for 5 d before harvest for 10× genomics single cell RNA sequencing analysis. As shown in FIG. 1, known RGC genes (Pou4f1 and Elavl4) were mainly expressed on clusters 3, 5 and 6. Pou2f2 and Tppp3 were observed in the same clusters as known RGC genes were. Pou2f2 and Tppp3 expression was reduced by GDF11 treatment, which was known to suppress RGC differentiation (Pou4f1). These data demonstrate that these two genes are potential novel RGC markers. Red arrow indicates GDF11 treatment group at cluster 3.

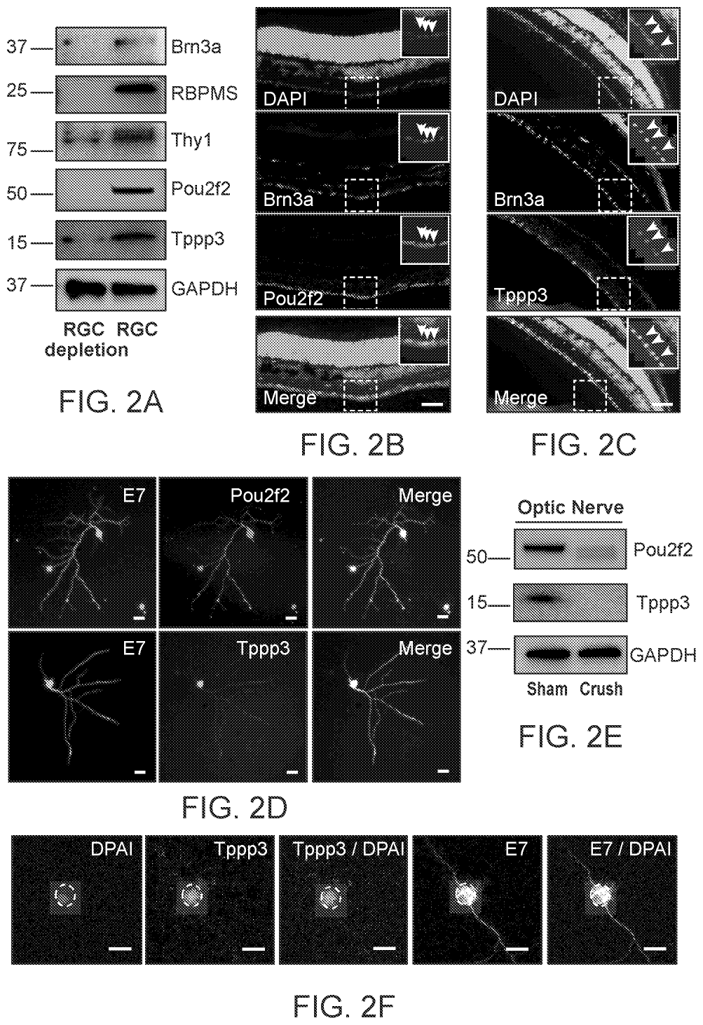

Pou2f2 and Tppp3 are expressed in mouse RGC in vitro and in vivo. (A) RGC and RGC depletion culture from P2 mouse retinas were collected for Western blot probing with RGC marker Brn3a, RBPMS, Thy1, Pou2f2 and Tppp3. (B-C) Brn3a, Pou2f2, Tppp3 and nucleus (DPAI) were probed in the adult mouse retinas, detected by immunofluorescence. (D) Pou2f2, Tppp3 and E7 were probed in the P2 mouse RGC, detected by immunofluorescence. (E) Normal (Sham) and injured (7 d after crush) mouse optic nerves were collected for Western blot probing with Pou2f2, Tppp3 and GAPDH (loading control). (F) Tppp3, E7 and nucleus (DPAI) were probed in the P2 mouse RGC, detected by immunofluorescence. As shown in FIG. 2, in vivo, Pou2f2 (B) and Tppp3 (C) were detected on the ganglion cell layer and arrows indicate the Brn3a co-labeling cells. (D) Immunostainging showed that Pou2f2 expresses in the soma and neurite of RGC but Tppp3 expression is very low in the neurite of RGC, confirmed with pIll-Tubulin (E7, neuronal marker) co-labeling. (E) Pou2f2 and Tppp3 proteins can be detected in optic never and were significant reduced by optic nerve crush. (F) Tppp3 mainly expresses in the soma of RGC. DAPI indicates nucleus. These data confirm that these two genes express in RGC.

Pou2f2 and Tppp3 regulate RGC differentiation and axon outgrowth. Pou2f2 and Tppp3 were observed in the developmental retinas from embryonic day 12 (E12) to postnatal day 0 (P0) by RNA hybridization (RNAscope) and qPT-PCR. (C) Pou2f2 and Tppp3 were overexpressed in ex vivo E14 retina culture by AAVs infection. (D) Pou2f2 and Tppp3 were genetic knockdown in ex vivo E14 retina culture by shRNA AAVs infection. (E) Pou2f2 and Tppp3 were overexpressed in P2 RGC culture by AAVs infection. (F) Pou2f2 and Tppp3 were knockdown in P2 RGC culture by AAVs infection.

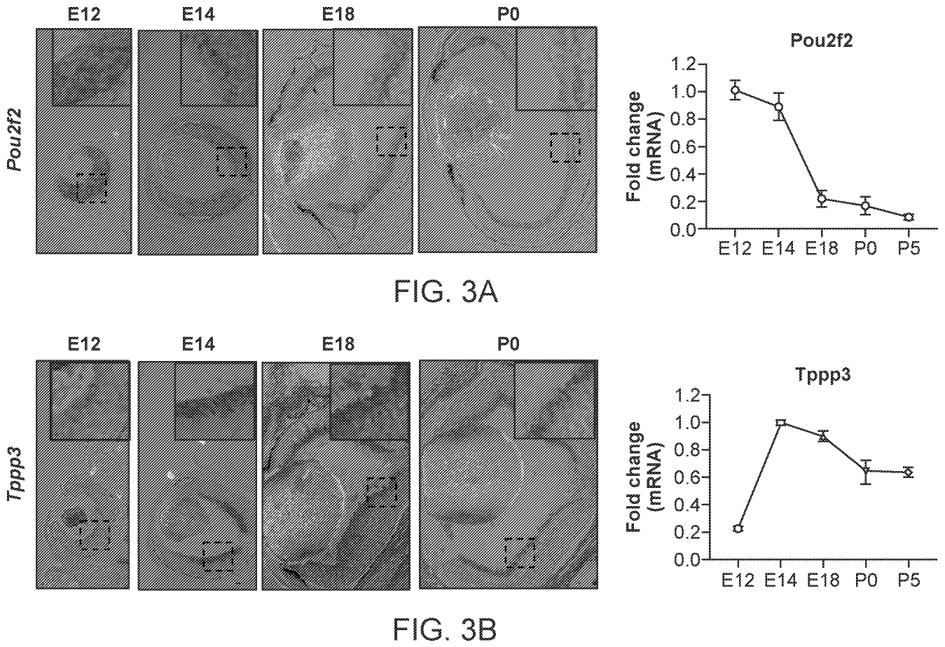

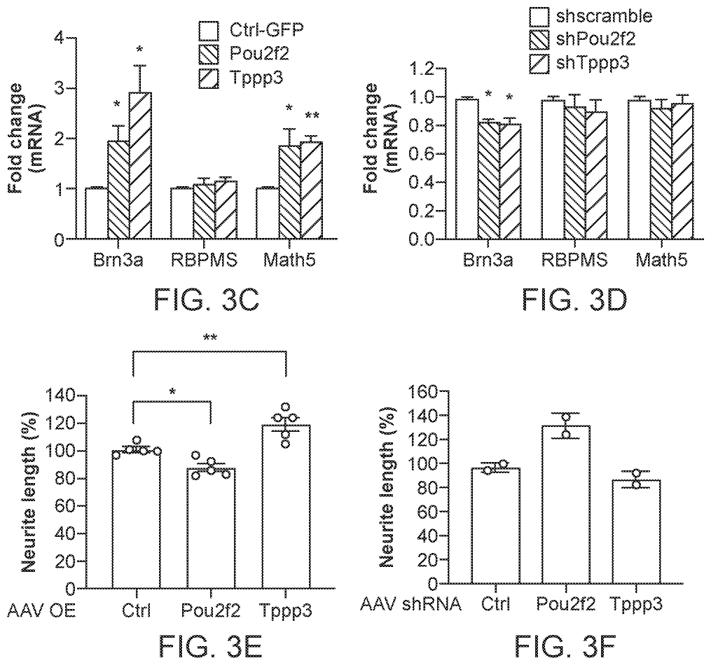

As shown in FIG. 3, (A) Messenger RNA of Pou2f2 starts to express on mouse retina on embryonic day 12 (E12) and keeps present at least till postnatal day 0 (P0). Total RNA of Pou2f2 expresses from E12 and is gradually decrease till P5. (B) Messenger RNA of Tppp3 starts to express on mouse retina on embryonic day 12 (E12) and keeps present at least till postnatal day 0 (P0). Total RNA of Tppp3 expresses from E12 and reach its peak at E14 and then gradually decrease till P5. (C) Exogenous expression of Pou2f2 and Tppp3 increases RGC marker Brn3a and Math5expression. (D) Genetic knockdown of Pou2f2 and Tppp3 reduces Brn3a expression. (E) Exogenous expression of Pou2f2 and Tppp3 slightly decreases and increases RGC axon outgrowth, respectively. (F) Genetic knockdown of Pou2f2 and Tppp3 slightly increases and decreases RGC axon outgrowth, respectively. These data indicate that these two genes regulate RGC differentiation.

1. A method of treating glaucoma or other optic neuropathies, the method comprising:
delivering to a retinal ganglion cell in the eye of a subject, a composition comprising a polynucleotide sequence that: (i) encodes one or both of Pou2f2 and Tppp3; (ii) is a fragment of one or both of Pou2f2 and Tppp3 sequences, or (iii) is complementary to one or both of Pou2f2 and Tppp3 sequences;
thereby preventing or reducing glaucoma or other optic neuropathies.

2. The method of claim 1, wherein the Pou2f2 or Tppp3 is human Pou2f2 or human Tppp3.

3. The method of claim 1, wherein the composition is administered intravitreally, subretinally, subconjunctivally, or intravenously.

4. The method of claim 1, wherein the subject is a human.

5. The method of claim 1, wherein the polynucleotide sequence is in operable linkage with a human cytomegalovirus immediate early enhancer/promoter.

6. The method of claim 1, wherein the polynucleotide encodes a functional human Pou2f2 protein.

7. The method of claim 1, wherein the polynucleotide encodes a functional human Tppp3 protein.

8. The method of claim 1, wherein the polynucleotide encodes a fragment of a human Pou2f2 protein.

9. The method of claim 1, wherein the polynucleotide encodes a fragment of a human Tppp3 protein.

10. The method of claim 1, wherein the polynucleotide comprises an siRNA, shRNA or anti-sense RNA of a human Pou2f2 sequence.

11. The method of claim 1, wherein the polynucleotide comprises an siRNA, shRNA or anti-sense RNA of a human Tppp3 sequence.

12. The method of claim 1, wherein the polynucleotide comprises a guide RNA specific for human Pou2f2.

13. The method of claim 1, wherein the polynucleotide comprises a guide RNA specific for human Tppp3.

14. The method of claim 1, wherein the polynucleotide sequence further comprises a woodchuck hepatitis virus posttranslational regulatory element.

15. The method of claim 1, wherein the polynucleotide sequence encoding further comprises a bovine growth hormone polyadenylation signal.

16. The method of claim 1, wherein the polynucleotide sequence further comprises AAV2 inverted terminal repeats.

17. The method of claim 1, wherein the composition further comprises a pharmaceutically acceptable diluent, excipient, vehicle, or carrier.

18. The method of claim 1, wherein the composition further comprises a second therapeutic agent for the treatment of glaucoma and other optic neuropathies.


SEQUENCE LISTING (2 sequences)

SEQ ID NO: 1 - DNA, 1021 bp, Homo sapiens
agtcccacccgggagccggcagggagcggagctgcggagccgcctggtctcccgcgtccatcggtccattcctgcgtcgttctgtccttccgaacgcacacttcaggagcagccgcgagggtggcatggcagcgagcacagacatggctgggctggaggagagcttccgcaagtttgccatccatggtgaccccaaggccagtgggcaagagatgaatggcaagaactgggccaagctgtgcaaggactgcaaggtggctgacggaaagtccgtgacagggaccgatgtggacatcgtcttctccaaagtcaaggggaagtctgctcgggtcatcaactatgaggagttcaagaaggccctggaagagctggcgaccaagagattcaaggggaagagcaaggaggaggccttcgatgccatctgccagctggtggcaggcaaagagccagccaatgtgggcgtcactaaagcaaaaacagggggtgctgtagaccggctgacggacaccagcagatacacgggctcccacaaggagcgcttcgatgagagcggcaagggcaagggcattgcgggacggcaggacatcctggacgacagtggctacgtgagcgcctacaagaatgcaggcacctacgatgccaaggtgaagaagtgaggcttgggaagaccgccctgccaagtgcggctgcccctgccagaggctcaggcctgggtctaaggggcacgtggagcaagagatcctggtcccctccctgctggacctgccacccagagcttcctgcctagtcccactgggctggcccaccaggcctctgacccaggctgctctgcggccccttcctcctcctcttcctgctccaacttctgtccacctggggacagtctgtgcctgtagcctcatgaccccaacccagccccaggcatggctaacccctgactgcttgcctcatatttaagctgctgctctggccaagtgcctaattttaacccagacctcaataaagacaccttttgtaccaa

SEQ ID NO: 2 - protein, 176 aa, Homo sapians
MAASTDMAGLEESFRKFAIHGDPKASGQEMNGKNWAKLCKDCKVADGKSVTGTDVDIVFSKVKGKSARVINYEEFKKALEELATKRFKGKSKEEAFDAICQLVAGKEPANVGVTKAKTGGAVDRLTDTSRYTGSHKERFDESGKGKGIAGRQDILDDSGYVSAYKNAGTYDAKVKK